(19) United States
(12) Patent Application Publication
(10) Pub. No.: US 2026/0130958 A1
(43) Pub. Date: May 14, 2026

(54) COMPOSITION FOR USE IN THE TREATMENT AND/OR PREVENTION OF DEGENERATIVE DISEASES OF THE EYE

(71) Applicant: SIGNIFER SL, Madrid (ES)

(72) Inventors: Jordi MONÉS CARILLA, Sant Cugat (ES); Maria Jesús GUARDIA ALBA, Granada (ES); Juan MOSCOSO DEL PRADO UCELAY, Tres Cantos (ES)

(21) Appl. No.: 19/121,051

(22) PCT Filed: Oct. 13, 2023
(86) PCT No.: PCT/EP2023/078517

§ 371 Date: Apr. 14, 2025

(30) Foreign Application Priority Data
Oct. 14, 2022 (ES) P202230887

(51) Int. Cl. A61K 36/064; A61K 9/00; A61K 35/742; A61K 35/745; A61K 35/747; A61K 45/06; A61P 27/02

(52) U.S. Cl. CPC A61K 36/064; A61K 9/0053; A61K 35/742; A61K 35/745; A61K 35/747; A61K 45/06; A61P 27/02

(57) ABSTRACT

A composition of oral administration for use in the prevention or and treatment degenerative diseases of the eye that it comprises lysates of probiotic micro-organisms in the form of a dry powder of the genus Bacillus, Lactobacillus, Streptococcus, Saccharomyces and Bifidobacterium.

### TECHNICAL FIELD OF THE INVENTION

The present invention describes a composition of oral administration to prevent and treat degenerative diseases of the eye characterized in that it comprises lysates of probiotic microorganisms in the form of dry powder. Therefore, the present invention is included in the technical field of therapies for degenerative diseases of the eye, particularly age-related macular degeneration (AMD).

### BACKGROUND

The average lifespan of the population has increased with the improvement of medical care and the modernization of society. With it, age-related diseases have also increased, making it increasingly evident that aging is an important factor in the development of degenerative diseases. These diseases not only cause central vision loss, but also include those that cause total vision loss such as inherited degenerative diseases of the retina. Among the diseases that are known to be associated with the degeneration of the eye, age-related macular degeneration (AMD), glaucoma, dry eye, etc. stand out. All of them negatively affect the quality of life of the sufferer and health costs. In many cases the mechanisms of the disease are not known although the people with the highest risk of developing them are the elderly.

AMD is the leading cause of blindness in the over-65-year-old population in industrialized countries. It affects 30-50 million people and despite the introduction of new therapies and prevention, it is expected to increase 10-fold by 2040. Its prevalence increases with age and shares common characteristics with other diseases such as Alzheimer's due to the presence of abnormal extracellular deposits associated with neuronal degeneration, drusen and plaques, respectively.

Macular degeneration is a disease of the eye, whose most frequent form is the atrophic or dry form, which appears because with age the retina decreases in thickness and the cells of the macula deteriorate, replacing viable cells with trophic areas that when extended can reach the center of the retina, which leads to a slow and gradual loss of vision. Once dry macular degeneration reaches the advanced stage, no treatment can prevent vision loss.

Although the exact pathogenesis of AMD is not known, exposure to risk factors such as age, genetics, lifestyle, environmental factors and diet is considered to influence its progression. Its development may be due to advanced age and exposure to environmental factors that induce high levels of oxidative stress damaging the macula. This damage causes inflammation inducing a vicious cycle that together causes vision loss. As a defense, the eye has a system of antioxidants formed mainly by vitamins, enzymes and carotenoids that have a synergistic effect and protect themselves in the process of destruction of free radicals. Vitamins C and E, and the carotenoids lutein and zeaxanthin are the most important. In addition, in the retina there is a large proportion of polyunsaturated fatty acids and photosensitive compounds that are highly susceptible to oxidation. These may be involved in the permeability, thickness, fluidity and other properties of the photoreceptors of the membrane, their insufficiency being linked to changes in the function of the retina. With age, the antioxidant system deteriorates due to damage to cellular structures, while the generation of free radicals increases and leads to lipid peroxidation. These peroxides interact with proteins, nucleic acids, and other components that affect the structure of the eye and its function. Degeneration of photoreceptors and accumulation of oxidized metabolites can lead to drusen formation and alterations in retinal pigment (RPE) that can evolve into AMD.

Because the increase in oxidative damage is one of the main factors, it seems logical to think that the contribution of antioxidants can be beneficial to prevent or slow down the progress of AMD. Diet is the main source of antioxidants providing vitamins, carotenoids, essential fatty acids and trace elements that are usually sufficient for healthy individuals. However, diet alone is not enough to provide the levels needed to protect the eye. In order to restore balance, antioxidant supplements have been proposed as therapy to slow the progression of AMD. One such study that has been conducted, “Age-Related Eye Disease Studies (AREDS) 1 and 2,” has shown that nutritional supplementation with antioxidants and micronutrients can effectively reduce progression to advanced forms of AMD by 28% over a 5-year period in patients over 55 years of age (7). This is currently the most common pre-registration for early and intermediate AMD.

There are multiple studies and documentations on techniques and methods for the treatment of macular degeneration diseases. Currently, one of the treatments for patients with AMD is intravitreal injections of anti-VEGF drugs, which is the antivascular endothelial growth factor. However, although this treatment is highly effective, it has the technical limitation of being a tedious treatment, that requires the intervention of a specialist in consultation and that can be painful for the patient.

In other degenerative diseases such as glaucoma, it seems that the mechanism causing vision loss is a consequence of damage to the optic nerve. Most treatments are aimed at controlling intraocular pressure with drugs and surgery, and when treatment is not effective, surgical treatments are used. In any case, the damage caused is not reversible.

Also in hereditary diseases, such as Stargardt's disease, there is an underlying inflammatory problem that when treated could improve the prognosis or slow down the disease.

That is why, faced with the technical problem posed by current treatments, the sector is actively working to develop treatments and compositions that can be administered orally to patients. Recently, some studies seem to indicate that the interaction of the microbiota with the eye can influence the evolution of the disease.

As proof of this, the following patent documents are cited.

The international patent application WO2022125925A describes methods for treating dry age-related macular degeneration (dry AMD), which includes administering to a subject in need a therapeutically effective amount of a compound or pharmaceutical composition. This method describes a pharmaceutical formulation.

U.S. Pat. No. 8,603,522B2 describes a daily nutritional or dietary supplement composition that strengthens and promotes retinal health through the prevention, stabilization, reversal and/or treatment of early-age-related macular degeneration. macular degeneration. The ingredients of the composition of the daily nutritional or dietary supplement include vitamin C, vitamin E, lutein, zinc and copper. The ingredients are preferably provided in the form of tablets suitable for oral ingestion.

The international patent application WO2021224679A1 concerns, in part, methods to prevent or treat macular degeneration in a subject by co-administering an enzyme superoxide dismutase and the probiotic Bacillus sp. spores, especially a mutant strain of Bacillus amyloliquefaciens GF423 or GF424. The present invention also provides pharmaceutical and/or food compositions comprising an enzyme superoxide dismutase and the probiotic Bacillus sp. spores.

It is also worth mentioning the Spanish patent applications P201930242 and P201930280, patent applications of the inventors, whose content is incorporated here for reference, and which describe a modulating composition of the human intestinal microbiome obtained from lysates of probiotic microorganisms and their method of obtainment, as well as a food supplement comprising the composition, which are useful in the prevention and treatment of disorders caused, or at least encouraged, by intestinal dysbiosis of the microbiota in humans.

However, none of the documents that are in the state of the art disclose a composition such as that described in this document, that is composed of dry bacterial lysates that allows the reduction and prevention of macular degeneration.

Therefore, the present invention aims to solve the problems of the prior art by using a composition for oral administration obtained from lysates of probiotic microorganisms for use in the treatment of macular degeneration in humans.

### BRIEF DESCRIPTION OF THE INVENTION

In a first aspect, the present invention relates to a composition for oral administration for use in the prevention and/or treatment of degenerative diseases of the eye, characterized in that it comprises lysates of probiotic microorganisms in the form of dry powder that are an amount in percentage by weight with respect to the total comprised between:
15% and 20% of bacterial lysates of the genus Bacillus;
15% and 35% of bacterial lysates of the genus Lactobacillus;
1.5% and 8% of bacterial lysates of the genus Streptococcus;
45% and 60% of bacterial lysates of the genus Saccharomyces; and
1.5% and 8% of bacterial lysates of the genus Bifidobacterium.

In a second aspect, the invention relates to the use of the composition object of the invention as a food supplement to prevent and treat degenerative diseases of the eye. In a preferred aspect, it refers to the use of the composition object of the invention as a food supplement for the prevention of macular degeneration by age.

In a third aspect, the invention relates to the composition that is defined herein for use as a medicament.

In a fourth aspect, the invention relates to the composition that is defined herein for use in the treatment of macular degeneration.

In a fifth aspect, the invention relates to a pharmaceutical composition comprising an effective pharmaceutical amount of the composition, in accordance with the first aspect of the invention, and a pharmaceutically acceptable excipient.

### BRIEF DESCRIPTION OF THE FIGURES

To complement the description and help to gain a better understanding of the invention, we have adjoined as an integral part of said description, a set of figures where, for illustrative and non-limiting purposes, the experimental results of a pilot study, described in example 3 of this document, have been presented.

FIG. 1. Progression of atrophy at 12 and 18 months for patient C1.

FIG. 2. Example of the change in trend of the evolution of atrophy for patient C1.

FIG. 3. Example of the evolution of atrophy for patient C6 without treatment.

FIG. 4: Evolution of the area in SQRT 12 months before and after starting treatment, BSL. (−26.58% decrease) of the 9 patients treated with the postbiotic composition of the invention. Every line represents an eye of a patient.

FIG. 5: Evolution of the area in SQRT 12 months before and after BSL, without treatment. (+4.5% increase) of the 9 patients without supplementation. Every line represents an eye of a patient.

### DETAILED DESCRIPTION OF THE INVENTION

As indicated in the previous sections, the inventors have detected the need to develop a composition that is administered to patients suffering from degenerative diseases of the eye, that is easy to administer and that does not present toxicity.

Based on this, they have developed a composition that is administered orally, comprising lysates of microorganisms accompanied by other essential components for this composition, and that has turned out to be strikingly beneficial for patients who have degenerative diseases of the eye.

Thus, in a first aspect, the present invention relates to a composition for oral administration to prevent and treat degenerative diseases of the eye, characterized in that it comprises lysates of probiotic microorganisms in the form of dry powder that are an amount in percentage by weight with respect to the total comprised between:
15% and 20% of bacterial lysates of the genus Bacillus;
15% and 35% of bacterial lysates of the genus Lactobacillus;
1.5% and 8% of bacterial lysates of the genus Streptococcus;
45% and 60% of bacterial lysates of the genus Saccharomyces; and
1.5% and 8% of bacterial lysates of the genus Bifidobacterium.

In the present specification, “postbiotic composition of the invention”, composition for oral administration” “postbiotics” are synonyms and can be used can be used interchangeably.

In the context of the present invention, bacterial lysates are understood as the product obtained after the process of cultivating, and subsequently, mechanically or chemically break up said bacterial cells in order to obtain a product with bacterial fragments, as well as all the components comprised therein. Likewise, in this document, it is indicated that these are dry bacterial lysates, since, thanks to their method of obtainment, these lysates are subsequently subjected to drying techniques, in such a way that said dry bacterial lysates are in the form of powder, also referred to in this document as dry powder.

In this document, degenerative eye disease is understood as 1) an eye disorder that progressively destroys central and/or peripheral vision, which can lead in very few years to legal blindness or total blindness, or 2) irreversible degenerative processes that cause very poor quality of life to patients, making even the most basic tasks extremely difficult. Within the first type, age-related macular degeneration, inherited degenerative diseases of the retina, glaucoma and uveitis or intraocular inflammatory diseases stand out. Within the second type, mainly dry eye and ocular surface problems.

Likewise, in the context of the present invention, the term probiotic microorganisms defines those live microorganisms, both bacteria and yeasts, which when consumed provide health benefits.

In preferred embodiments of the present invention, the composition object of the invention comprises bacterial lysates of the genus Bacillus in an amount in percentage by weight between 15% to 20%. In preferred embodiments, the amount of bacterial lysates of the genus Bacillus can be 15%, 16%, 17%, 18%, 19% or 20%.

In preferred embodiments of the present invention, the composition object of the invention comprises bacterial lysates of the genus Lactobacillus in an amount in percentage by weight between 15% to 35%, preferably, between 18%-30%, and even more preferably between 20%-28%. In preferred embodiments, the amount of bacterial lysates of the genus Lactobacillus can be 15%, 16%, 17%, 18%, 19%, 20%, 21%, 22%, 23%, 24%, 25%, 26%, 27%, 28%, 29% or 30%.

In preferred embodiments of the present invention, the composition object of the invention comprises bacterial lysates of the genus Streptococcus in an amount in percentage by weight between 1.5% to 8%, preferably between 2%-7%, and even more preferably between 3%-6%. In preferred embodiments, the amount of bacterial lysates of the genus Streptococcus can be 1.5%, 1.6%, 1.7%, 1.8%, 1.9%, 2%, 2.1%, 2.2%, 2.3%, 2.4%, 2.5%, 2.6%, 2.7%, 2.8%, 2.9% or 3%. In other preferred embodiments, the amount of bacterial lysates of the genus Streptococcus can be 3.5%, 4%, 4.5%, 5%, 5.5%, 6%, 6.5%, 7%, 7.5% or 8%.

In preferred embodiments of the present invention, the composition object of the invention comprises bacterial lysates of the genus Saccharomyces in an amount in percentage by weight between 45% to 60%, preferably, between 50%-58%, and even more preferably between 54%-56%. In preferred embodiments, the amount of bacterial lysates of the genus Saccharomyces can be 45%, 46%, 47%, 48%, 49%, 50%, 51%, 52%, 53%, 54%, 55%, 56%, 57%, 58%, 59% or 60%.

In preferred embodiments of the present invention, the composition object of the invention comprises bacterial lysates of the genus Bifidobacterium in an amount in percentage by weight between 1.5% to 8%, preferably between 2%-7%, and even more preferably between 3%-6%. In preferred embodiments, the amount of bacterial lysates of the genus Bifidobacterium can be 1.5%, 1.6%, 1.7%, 1.8%, 1.9%, 2%, 2.1%, 2.2%, 2.3%, 2.4%, 2.5%, 2.6%, 2.7%, 2.8%, 2.9% or 3%. In other preferred embodiments, the amount of bacterial lysates of the genus Streptococcus can be 3.5%, 4%, 4.5%, 5%, 5.5%, 6%, 6.5%, 7%, 7.5% or 8%.

In preferred embodiments of the present invention, the bacterial lysates of the genus Bacillus are of the species that are selected from the group consisting of Bacillus coagulans, Bacillus licheniformis, Bacillus mesentericus, Bacillus subtilis, Bacillus clausii, Bacillus paralicheniformis, and combinations thereof, preferably Bacillus coagulans, Bacillus licheniformis, Bacillus mesentericus, and Bacillus subtilis, or Bacillus licheniformis and Bacillus subtilis

In other embodiments, the bacterial lysates of the genus Lactobacillus are of the species that are selected from the group consisting of Lactobacillus acidophilus, Lactobacillus bulgaricus, Lactobacillus casei, Lactobacillus fermentum, Lactobacillus reuteri, Lactobacillus rhamnosus, Lactobacillus paracasei, Lactobacillus helveticus, Lactobacillus johnsonii, Lactobacillus plantarum, Lactobacillus salivarius, Lactobacillus gasseri, Lactobacillus kefiri, and combinations thereof, preferably Lactobacillus acidophilus, Lactobacillus bulgaricus, Lactobacillus casei, Lactobacillus fermentum, Lactobacillus reuteri, and Lactobacillus rhamnosus or Lactobacillus acidophilus, Lactobacillus casei, Lactobacillus fermentum, Lactobacillus reuteri, and Lactobacillus rhamnosus.

In addition, in other embodiments, the bacterial lysates of the genus Streptococcus are of the species that are selected from the group consisting of Streptococcus thermophilus, Streptococcus salivarius, and combinations thereof, preferably Streptococcus thermophilus.

In other particular embodiments of the present invention, the bacterial lysates of the genus Saccharomyces are of the species that are selected from the group consisting of Saccharomyces cerevisiae, Saccharomyces boulardii, and combinations thereof, preferably Saccharomyces cerevisiae.

In other preferred embodiments of the present invention, the bacterial lysates of the genus Bifidobacterium are of the species that are selected from the group consisting of Bifidobacterium bifidum, Bifidobacterium lactis, Bifidobacterium breve, Bifidobacterium longum, Bifidobacterium animalis subsp. lactis, Bifidobacterium infantis, Bifidobacterium animalis, and combinations thereof, preferably Bifidobacterium bifidum and Bifidobacterium lactis.

In a particular embodiment of the invention, the composition comprises lysates of probiotic microorganisms in an amount in percentage by weight of the total lysates of the composition:
approximately 15% to 18% of bacterial lysates of the genus Bacillus;
approximately 3% to 7% of bacterial lysates of the genus Bifidobacterium;
approximately 17% to 30% of bacterial lysates of the genus Lactobacillus;
approximately 50% to 57% of yeast lysates of the genus Saccharomyces;
approximately 2% to 5% of bacterial lysates of the genus Streptococcus.

### This Composition is Called Composition “A”

In another particular embodiment of the invention, the composition comprises lysates of probiotic microorganisms in an amount in percentage by weight on the total lysates of the composition:
approximately 15% to 17% of bacterial lysates of the genus Bacillus;
approximately 4.6% to 6% of bacterial lysates of the genus Bifidobacterium;
approximately 20% to 25% of bacterial lysates of the genus Lactobacillus;
approximately 54% to 56% of yeast lysates of the genus Saccharomyces;
approximately 2.8% to 3.2% of bacterial lysates of the genus Streptococcus.

### This Composition is Called Composition “B”

In another particular embodiment of the invention, the composition comprises lysates of probiotic microorganisms in an amount in percentage by weight on the total lysates of the composition:
approximately 16% of bacterial lysates of the genus Bacillus;
approximately 5% of bacterial lysates of the genus Bifidobacterium;
approximately 21% of bacterial lysates of the genus Lactobacillus;
approximately 55% of yeast lysates of the genus Saccharomyces;
approximately 3% of bacterial lysates of the genus Streptococcus.

### This Composition is Called Composition “C”

In a particular embodiment of the invention, the composition comprises lysates of probiotic microorganisms in an amount in percentage by weight on the total lysates of the composition:
approximately 15% to 20% bacterial lysates of Bacillus licheniformis and Bacillus subtilis; and, optionally Bacillus mesentericus and Bacillus coagulans
approximately 1.5% to 8% of bacterial lysates of Bifidobacterium bifidum and Bifidobacterium lactis;
approximately 15% to 35% of bacterial lysates of Lactobacillus acidophilus, Lactobacillus casei, Lactobacillus fermentum, Lactobacillus reuteri, and Lactobacillus rhamnosus; and, optionally Lactobacillus bulgaricus;
approximately 45% to 60% of yeast lysates of Saccharomyces cerevisiae;
approximately 1.5% to 8% of bacterial lysates of Streptococcus thermophilus.

### This Composition is Called Composition “D”

In a particular embodiment of the invention, the composition comprises lysates of probiotic microorganisms in an amount in percentage by weight of the total lysates of the composition:
approximately 15% to 18% of bacterial lysates Bacillus licheniformis and Bacillus subtilis; and, optionally Bacillus mesentericus and Bacillus coagulans;
approximately 3% to 7% of bacterial lysates of Bifidobacterium bifidum and Bifidobacterium lactis;
approximately 17% to 30% of bacterial lysates of Lactobacillus acidophilus, Lactobacillus casei, Lactobacillus fermentum, Lactobacillus reuteri, and Lactobacillus rhamnosus; and, optionally Lactobacillus bulgaricus;
approximately 50% to 57% of yeast lysates of Saccharomyces cerevisiae;
approximately 2% to 5% of bacterial lysates of Streptococcus thermophilus.

### This Composition is Called Composition “E”

In another particular embodiment of the invention, the composition comprises lysates of probiotic microorganisms in an amount in percentage by weight on the total lysates of the composition:
approximately 15% to 17% of bacterial lysates of Bacillus licheniformis and Bacillus subtilis; and, optionally Bacillus mesentericus and Bacillus coagulans;
approximately 4.6% to 6% of bacterial lysates of Bifidobacterium bifidum and Bifidobacterium lactis;
approximately 20% to 25% of bacterial lysates of Lactobacillus acidophilus, Lactobacillus casei, Lactobacillus fermentum, Lactobacillus reuteri, and Lactobacillus rhamnosus; and, optionally Lactobacillus bulgaricus;
approximately 54% to 56% of yeast lysates of Saccharomyces cerevisiae;
approximately 2.8% to 3.2% of bacterial lysates of Streptococcus thermophilus.

### This Composition is Called Composition “F”

In another particular embodiment of the invention, the composition comprises lysates of probiotic microorganisms in an amount in percentage by weight on the total lysates of the composition:
Bacillus licheniformis 1% to 20% and Bacillus subtilis 1% to 12%; and, optionally Bacillus mesentericus 1% to 10% and Bacillus coagulans 0.1% to 10%
Bifidobacterium lactis: 0.3% to 10% and Bifidobacterium bifidum: 0.1% to 8%;
Lactobacillus acidophilus 0.5% to 22%, Lactobacillus casei 0.1% to 12%, Lactobacillus fermentum 0.1% to 12%, Lactobacillus reuteri 0.1% to 6%, and Lactobacillus rhamnosus 0.1% to 6%; and, optionally Lactobacillus bulgaricus 0.2% to 10%;
Saccharomyces cerevisiae: 35% to 75%
Streptococcus thermophilus: 0.1% to 8%

### This Composition is Called Composition “G”

In another particular embodiment of the invention, the composition comprises lysates of probiotic microorganisms in an amount in percentage by weight on the total lysates of the composition:
Bacillus licheniformis 2% to 15% and Bacillus subtilis 2% to 10%; and, optionally Bacillus mesentericus 2% to 8% and Bacillus coagulans 0.2% to 8%
Bifidobacterium lactis: 0.5% to 8% and Bifidobacterium bifidum: 0.5% to 7%;
Lactobacillus acidophilus 1% to 20%, Lactobacillus casei 0.5% to 10%, Lactobacillus fermentum 0.3% to 10%, Lactobacillus reuteri 0.5% to 5%, and Lactobacillus rhamnosus 0.3% to 5%; and, optionally Lactobacillus bulgaricus 0.5% to 8%;
Saccharomyces cerevisiae: 40% to 70%
Streptococcus thermophilus: 0.3% to 6%

### This Composition is Called Composition “H”

In another particular embodiment of the invention, the composition comprises lysates of probiotic microorganisms in an amount in percentage by weight on the total lysates of the composition:
Bacillus licheniformis 3% to 11% and Bacillus subtilis 3% to 9%; and, optionally Bacillus mesentericus 3% to 7% and Bacillus coagulans 0.3% to 7%
Bifidobacterium lactis: 1% to 7% and Bifidobacterium bifidum: 0.8% to 6%;
Lactobacillus acidophilus 2% to 18%, Lactobacillus casei 0.5% to 9%, Lactobacillus fermentum 0.5% to 9%, Lactobacillus reuteri 0.7% to 4.5%, and Lactobacillus rhamnosus 0.5% to 4.5%; and, optionally Lactobacillus bulgaricus 0.7% to 7%;
Saccharomyces cerevisiae: 43% to 68%
Streptococcus thermophilus: 0.5% to 5.5%

### This Composition is Called Composition “I”

In another particular embodiment of the invention, the composition comprises lysates of probiotic microorganisms in an amount in percentage by weight on the total lysates of the composition:
Bacillus licheniformis 4% to 11% and Bacillus subtilis 4% to 8%; and, optionally Bacillus mesentericus 3% to 6% and Bacillus coagulans 0.4% to 6%
Bifidobacterium lactis: 1.5% to 6% and Bifidobacterium bifidum: 0.8% to 5%;
Lactobacillus acidophilus 2.5% to 15%, Lactobacillus casei 0.5% to 8%, Lactobacillus fermentum 1% to 8%, Lactobacillus reuteri 0.9% to 4%, and Lactobacillus rhamnosus 0.7% to 4%; and, optionally Lactobacillus bulgaricus 1% to 5%;
Saccharomyces cerevisiae: 45% to 65%
Streptococcus thermophilus: 1% to 5%

### This Composition is Called Composition “J”

In another particular embodiment of the invention, the composition comprises lysates of probiotic microorganisms in an amount in percentage by weight on the total lysates of the composition:
Bacillus licheniformis 4.5% to 10.5% and Bacillus subtilis 4.5% to 8%; and, optionally Bacillus mesentericus 3% to 6% and Bacillus coagulans 0.4% to 3%
Bifidobacterium lactis: 2% to 5.5% and Bifidobacterium bifidum: 0.8% to 3%;
Lactobacillus acidophilus 3% to 12%, Lactobacillus casei 0.6% to 7%, Lactobacillus fermentum 2% to 7%, Lactobacillus reuteri 1% to 3%, and Lactobacillus rhamnosus 1% to 4%; and, optionally Lactobacillus bulgaricus 2% to 5%;
Saccharomyces cerevisiae: 50% to 60%
Streptococcus thermophilus: 2% to 4%

### This Composition is Called Composition “K”

In a particular embodiment of the invention, the composition for use, comprises the lysates of probiotic microorganisms in the form of dry powder, in the amount as a percentage by weight of the total as disclosed in compositions “A”, “B”, “C”, “D” E”, “F”, “G”, “H”, “I”. “J” or “K”

In other particular embodiments of the present invention, the composition object of the invention may comprise another additional component or additive that is selected from the group consisting of Vitamin B1, Vitamin B2, Vitamin B3, Vitamin B6, Vitamin B9, Vitamin B12, Zeaxanthin, Hydroxytyrosol, Omega 3 fatty acids (DHA), Glutathione, Copper, Selenium, and combinations thereof.

Below, we indicate the quantity in which each of the additional components or additives that can optionally comprise the composition, in accordance with the first aspect of the present invention, can be included.

TABLE 1
List of additional components that may comprise the composition
object of the invention, and the quantities in particular,
preferred and most preferred embodiments.

| | Content in | | Content in | | Content in most |
|---|---|---|---|---|---|
| | particular | | preferred | | preferred |
| Component | embodiments | | embodiments | | embodiments |
| Vitamin B1 | 0.1-2 | mg | 0.5-1.5 | mg | 1.1 mg ± 10% |
| Vitamin B2 | 0.1-30 | mg | 5-25 | mg | 20 mg ± 10% |
| Vitamin B3 | 2-25 | mg | 4-20 | mg | 16 mg ± 10% |
| Vitamin B6 | 0.2-35 | mg | 5-30 | mg | 25 mg ± 10% |
| Vitamin B9 | 0.032-2 | mg | 0.1-1.5 | mg | 1 mg ± 10% |
| Vitamin B12 | 0.1-6 | μg | 0.4-4 | μg | 2.5 μg ± 10% |
| Vitamin C | 10-700 | mg | 50-600 | mg | 500 mg |
| Vitamin E | 1-400 | mg | 50-350 | mg | 300 mg |
| Zeaxanthin | 0.2-3 | mg | 0.4-2 | mg | 1.5 mg ± 10% |
| Hydroxytyrosol | 0.02-2 | mg | 0.1-1.5 | mg | 1 mg ± 10% |
| Omega 3 fatty | 23-250 | mg | 40-200 | mg | 150 mg ± 10% |
| acids (DHA) | | | | | |
| Glutathione | 0.1-2.5 | mg | 0.3-2 | mg | 1.5 mg ± 10% |
| Copper | 0.1-4 | mg | 1-30 | mg | 2 mg ± 10% |
| Selenium | 10-400 | μg | 50-350 | μg | 300 μg ± 10% |
| Zinc | 0.5-40 | mg | 1-30 | mg | 25 mg |
| Lutein | 0.5-20 | mg | 1-15 | mg | 10 mg |

The additional components present in the composition can be products to cover common deficiencies in vitamins that usually occur in humans from the age of 50 such as vitamins 1 and B3, which boost the normal energy performance of the metabolism and help maintain the correct function of the defense system.

The antioxidant components, such as hydroxytyrosol, a polyphenol present in the fruit of the olive tree, which protects against oxidative stress, or selenium which is a trace element necessary for antioxidant defenses, or glutathione that together with the enzymes superoxide dismutase and glutathione peroxidase are part of the endogenous system of the organism, further contribute to the protection against oxidative stress.

Minerals for bone maintenance and magnesium for muscle function, soluble fiber, omega 3 fatty acids that improve memory performance.

Finally, another component that should be highlighted is copper, which contributes to the maintenance of connective tissue in normal conditions.

In the context of the present invention, the method by which the composition object of the invention is obtained is also described.

The postbiotic is composed of lysates of probiotic microorganisms comprising species of the genera Bacillus, Lactobacillus, Streptococcus, Saccharomyces and Bifidobacterium.

Based on the fact that the composition object of the invention is in powder form, the composition, in accordance with the first aspect of the invention, can be presented in sealed sachets, in themselves conventional in the food and pharmaceutical industry.

Another form of presentation of the food supplement, in accordance with the invention, is in capsule form, such as conventional gelatin capsules, inside which the composition in powder form is contained.

In preferred embodiments of the present invention, the composition may comprise a relative abundance relative to the totality of microbial proteins of between 4% to 6% of proteins from bacterial lysates of the genus Bacillus in percentage by weight, preferably between 4.14% to 5.61%, and even more preferably between 4.21% to 5.27%. In preferred embodiments, the relative abundance of proteins from bacterial lysates of the genus Bacillus can be 4%, 4.20%, 4.40%, 4.60%, 4.80%, 5%, 5.20%, 5.40%, 5.60%, 5.80% or 6%.

In preferred embodiments of the present invention, the proteins that have been identified from bacterial lysates of the genus Bacillus are selected from the group consisting of Q65HF3, P04831, P04832, and combinations thereof.

In preferred embodiments of the present invention, the composition may comprise an abundance relative to the totality of microbial proteins of between 0.02% to 0.10% of proteins from bacterial lysates of the genus Bifidobacterium in percentage by weight, and preferably between 0.07% to 0.09%. In preferred embodiments, the relative abundance of proteins from bacterial lysates of the genus Bifidobacterium can be 0.02%, 0.03%, 0.04%, 0.05%, 0.06%, 0.07%, 0.08%, 0.09% or 0.10%.

In preferred embodiments of the present invention, the proteins that have been identified from bacterial lysates of the genus Bifidobacterium are selected from the group consisting of B8DTX9, B8DSQ4, and combinations thereof.

In preferred embodiments of the present invention, the composition may comprise an abundance relative to the totality of microbial proteins of at least 3% to 8.50% of proteins from bacterial lysates of the genus Lactobacillus in an amount in percentage by weight, preferably between 3.45% to 8.06%, and even more preferably between 4.84% to 5.45%. In preferred embodiments, the relative abundance of proteins from bacterial lysates of the genus Lactobacillus can be 3%, 3.50%, 4%, 4.50%, 5%, 5.50%, 6%, 6.50%, 7%, 7.50%, 8% or 8.50%.

In preferred embodiments of the present invention, the proteins that have been identified from the bacterial lysates of the genus Lactobacillus are selected from the group consisting of A0A0E2BRTT9, D8I1HB6, Q5FKM6, and combinations thereof.

In preferred embodiments of the present invention, the composition may comprise an abundance relative to the totality of microbial proteins between 70% to 99.00% of proteins from bacterial lysates of the genus Saccharomyces in an amount in percentage by weight, preferably between 73.85% to 98.5%, and even more preferably between 85% to 90.30%. In preferred embodiments, the relative abundance of proteins from bacterial lysates of the genus Saccharomyces can be 71%, 72%, 73%, 74%, 75%, 76%, 77%, 78%, 79%, 80%, 81%, 82%, 83%, 84%, 85%, 86%, 87%, 88% or 89%. In other preferred embodiments, the relative abundance of proteins from bacterial lysates of the genus Saccharomyces can be 90%, 90.5%, 91%, 91.5%, 92%, 92.5%, 93%, 93.5%, 94%, 94.5%, 95%, 95.5%, 96%, 96.5%, 97%, 97.5%, 98%, 98.5% or 99%.

In preferred embodiments of the present invention, the proteins that have been identified from bacterial lysates of the genus Saccharomyces are selected from the group consisting of A0A0L8VPY0, P00359, A0A0L8VN66, A0A0L8VP44, and combinations thereof.

In preferred embodiments of the present invention, the composition may comprise an abundance relative to the totality of microbial proteins between 0.15% to 0.90% of proteins from bacterial lysates of the genus Streptococcus in percentage by weight, preferably between 0.17% to 89%, and even more preferably between 0.33% to 66%. In preferred embodiments, the relative abundance of proteins from bacterial lysates of the genus Streptococcus can be 0.15%, 0.20%, 0.25%, 0.30%, 0.35%, 0.40%, 0.45%, 0.50%, 0.55%, 0.60%, 0.65%, 070%, 0.75%, 0.80%, 0.85% or 0.90%.

In preferred embodiments of the present invention, the proteins that have been identified from the bacterial lysates of the genus Streptococcus are selected from the group consisting of Q5M561, Q5M518, and combinations thereof.

To obtain the composition object of the invention, one begins with a set of cultures of the genera described in this document, which comprise a certain amount of Colony Forming Units (CFU). In the context of the present invention, a Colony Forming Unit is a term of microbiology. It is an indicator of the amount of live microorganisms present in a medium.

In particular embodiments of the present invention, one starts with a number of bacteria of the genus Bacillus comprising between 10.56% to 14.08% of CFU with respect to the total CFU of the composition, most preferably between 11.26% to 12.75% of CFU.

In particular embodiments of the present invention, one starts with a number of bacteria of the genus Lactobacillus comprising between 29.40% to 68.61% of CFU with respect to the total CFU of the composition, most preferably between 41.17% to 53.36% of CFU.

In particular embodiments of the present invention, one starts with a number of bacteria of the genus Streptococcus comprising between 5.83% to 31.07% of CFU with respect to the total CFU of the composition, most preferably between 11.65% to 18.15% of CFU.

In particular embodiments of the present invention, one starts with a number of bacteria of the genus Saccharomyces comprising between 17.48% to 23.30% of CFU with respect to the total CFU of the composition, most preferably between 21.36% to 22.45% of CFU.

In particular embodiments of the present invention, one starts with a number of bacteria of the genus Bifidobacterium comprising between 4.37% to 23.30% of CFU with respect to the total CFU of the composition, most preferably between 14.56% to 17.35%.

These probiotic bacteria are cultured under standard conditions, as set out in the culture protocols published by the Spanish Collection of Type Cultures (CECT), indicated for each of the bacterial species described in this document.

Once these microorganisms are cultured, they are subjected to a lysis procedure. The process of obtaining lysates consists in combining a non-mechanical method with a mechanical one. Firstly, the microbial cells undergo heat treatment. Each batch of viable cell culture undergoes a sterilization cycle in an autoclave at 121° C. for 20 to 30 minutes. This temperature denatures and coagulates the proteins by inactivating them. It also causes membrane damage, ribosome aggregation, DNA strand breakdown and enzyme inactivation. Once cold, they undergo a cell rupture treatment by sonication for a period of 15 to 20 min, at an output power of between 450 to 550 W, at an amplitude of between 38 to 43% and a period of 8 to 15 seconds of pause, obtaining a mass of lysed cells from the probiotic batch (Qsonica, Q500). This results in the breakage of intermolecular interactions and DNA fragmentation. The final solution is freeze-dried and ground to obtain a lysate of the powdery probiotic microorganism. The powder is stored in a cool environment away from heat.

In a second aspect, the invention relates to the use of the composition object of the invention as a food supplement for the prevention and/or delay of degenerative diseases of the eye. In preferred embodiments, degenerative diseases of the eye are selected from the group consisting of age-related macular degeneration, inherited degenerative diseases of the retina, glaucoma, uveitis or intraocular inflammatory diseases, dry eye, alterations of the ocular surface, and combinations thereof.

In a third aspect, the invention relates to the composition that is defined herein for use as a medicament.

In a fourth aspect, the invention relates to the composition that is defined herein for use in the treatment of degenerative diseases of the eye.

Degenerative diseases of the eye can be one or more of the following: Age-Related Macular Degeneration (AMD), Retinitis Pigmentosa, Glaucoma, Corneal Dystrophy, Cataracts, Stargardt's Disease, Leber's Disease, Keratoconus, Best's Disease, Optic Nerve Atrophy.

In a particular embodiment, the degenerative disease may be in one eye or in both eyes.

In a preferred embodiment, the degenerative eye disease is Age-Related Macular Degeneration (AMD), unless otherwise indicated, AMD can be both wet AMD and dry AMD at any stage of progression, such as early intermediate or advance, and having the drusen in any condition such as soft drusen, hard drusen, mixed drusen and reticular drusen.

In a fifth aspect, the invention relates to a pharmaceutical composition comprising an effective pharmaceutical amount of the composition, in accordance with the first aspect of the invention, and a pharmaceutically acceptable excipient.

In the context of the present invention, the expression “pharmaceutical composition” refers to a formulation that has been adapted to deliver a predetermined dose of one or more useful therapeutic agents to a cell, a group of cells, an organ or a tissue.

The term “effective pharmaceutical quantity”, as used herein, is understood as an amount capable of providing a therapeutic effect, and which can be determined by a person skilled in the art by commonly used means.

Also, in the context of the present invention, “pharmaceutically acceptable excipient” means a therapeutically inactive substance that is said to be used to incorporate the active ingredient and that is acceptable to the patient from a pharmacological/toxicological standpoint, and to the pharmaceutical chemist who manufactures it from a physical/chemical standpoint, with respect to composition, formulation, stability, patient acceptance and bioavailability.

In the context of the present invention, what is understood by food supplement is a food product whose purpose it is to supplement the normal diet and which consists of concentrated sources of nutrients or other substances that have a nutritional or physiological effect, in simple or combined form, which is marketed in dosed form, ie capsules, pills, tablets, pastilles and other similar forms, sachets of powders, ampoules of liquid, dropper bottles and other similar forms of liquids and powders to be taken in small unit quantities as defined in Directive 2002/46/EC of the European Parliament.

In a particular embodiment of the present invention, the composition for use in the treatment of macular degeneration comprises orally administering to a patient a dose at least twice a day. Also, for the treatment to be effective and to achieve the desired effect, this dose should be administered to the patient at least twice a day and a maximum of 6 times a day. Preferably, the administration of the dose to the patient is 3, 4 or 5 times a day, most preferably 3 times a day.

In the context of the present invention, a dose is defined as the amount of medicine containing the exact measure of active ingredient so that it is efficient, effective and safe for the patient, and solves the health problem for which it has been indicated.

Thus, in the context of the present invention, it has been determined that a dose contains an amount of the composition object of the invention of between 100 mg to 400 mg. In preferred embodiments, a dosage can comprise between 150 mg to 300 mg of the composition. In particular embodiments of the present invention, a dose may comprise 100 mg, 150 mg, 200 mg, 250 mg, 300 mg, 350 mg or 400 mg of the composition object of the invention.

In particular embodiments where the composition is presented in watertight sachets, this composition can be administered to the patient dissolved or suspended in a liquid, preferably in an aqueous liquid, and more preferably, in beverages such as fruit juices, milk, water. In addition, it can also be mixed with food such as yogurt, liquid yogurt, soups, purees, creams, or porridge. These foodstuffs have to be at optimum temperature to be consumed, and must never be heated after having added the composition object of the invention.

In a particular embodiment of the present invention, the food supplement described herein is administered orally to a patient with macular degeneration, one dose at least twice a day. Also, for the treatment to be effective and to achieve the desired effect, this dose should be administered to the patient at least twice a day and a maximum of 6 times a day. Preferably, the administration of the dose to the patient is 3, 4 or 5 times a day, most preferably 3 times a day.

In particular embodiments where the composition is presented in watertight sachets, said food supplement can be administered to the patient dissolved or suspended in a liquid, preferably in an aqueous liquid, and more preferably, in beverages such as fruit juices, milk, water. In addition, it can also be mixed with food such as yogurt, liquid yogurt, soups, purees, creams, or porridge. These foodstuffs have to be at optimum temperature to be consumed, and must never be heated after having added the composition object of the invention.

The composition object of the invention is the result of the combination of probiotic microorganisms after a process of growth and lysis, which generates an extract consisting of a mixture of metabolites, proteins, DNA fragments and other components, such as, for example, peptidoglycans, which dosed efficiently is able to alter and modify the microbiota of the host through several mechanisms so as to produce an activation of the immune system, reversing dysbiosis naturally.

The technical effect derived from the composition object of the invention is that it acts directly on the microbiota of patients with macular degeneration, modulating and improving said microbiota. Thanks to this restorative and modulating action, the inventors have confirmed that, strikingly, intervening in the microbiota of a patient with macular degeneration can significantly improve the state of this disease, slowing down this deterioration.

In this way the inventors have been able to verify that the oral administration of the composition object of the invention exerts an effect on an adjustment and reprogramming of the microbiota present in the intestinal tract of the patient that is not a local effect, since the whole organism is benefited by this modulation of the microbiota, thus becoming a systemic effect.

Therefore, the main advantages arising from the composition object of the invention are the following:
supplement and/or complement the physiological functions of the patients' microbiota;
the alterations that generate dysbiosis and produce a clinical impact on macular diseases of the human eye, particularly age-related macular degeneration (AMD), are restored;
the fact that the administration of the product is oral, makes the present composition and its striking effect present itself as a very advantageous alternative compared to the treatments currently used in medicine that have been mentioned above, such as the injection of a drug into the eyeball, since in addition to being painful this requires the intervention of a specialist;
The present composition includes lysates of microorganisms, that is, it does not present living organisms, such that the present composition is presented as a safe alternative, because it would avoid the possible dangers of colonizing organisms, and additionally it does not present the toxicity that a conventional drug can present.

Moreover, the invention refers to the following clauses:

1. A composition of oral administration to prevent and treat degenerative diseases of the eye, characterized in that it comprises lysates of probiotic microorganisms in the form of dry powder that are an amount, as a percentage by weight of the total, comprised between:
15% and 20% of bacterial lysates of the genus Bacillus;
15% and 35% of bacterial lysates of the genus Lactobacillus;
1.5% and 8% of bacterial lysates of the genus Streptococcus;
45% and 60% of bacterial lysates of the genus Saccharomyces; and
1.5% and 8% of bacterial lysates of the genus Bifidobacterium.

2. The composition, in accordance with clause 1, wherein the bacterial lysates of the genus Bacillus are of the species that are selected from the group consisting of Bacillus coagulans, Bacillus licheniformis, Bacillus mesentericus, Bacillus subtilis, Bacillus clausii, Bacillus paralicheniformis, and combinations thereof.

3. The composition, in accordance with clauses 1 or 2, wherein the bacterial lysates of the genus Bifidobacterium are of the species that are selected from the group consisting of Bifidobacterium bifidum, Bifidobacterium lactis, Bifidobacterium breve, Bifidobacterium longum, Bifidobacterium animalis subsp. lactis, Bifidobacterium infantis, Bifidobacterium animalis, and combinations thereof.

4. The composition, in accordance with any of clauses 1 to 3, wherein the bacterial lysates of the genus Lactobacillus are of the species that are selected from the group consisting of Lactobacillus acidophilus, Lactobacillus bulgaricus, Lactobacillus casei, Lactobacillus fermentum, Lactobacillus reuteri, Lactobacillus rhamnosus, Lactobacillus paracasei, Lactobacillus helveticus, Lactobacillus johnsonii, Lactobacillus plantarum, Lactobacillus salivarius, Lactobacillus gasseri, Lactobacillus kefiri, and combinations thereof.

5. The composition, in accordance with any of clauses 1 to 4, wherein the bacterial lysates of the genus Saccharomyces are of the species that are selected from the group consisting of Saccharomyces cerevisiae, Saccharomyces boulardii, and combinations thereof.

6. The composition, in accordance with any of clauses 1 to 5, wherein the bacterial lysates of the genus Streptococcus are of the species that are selected from the group consisting of Streptococcus salivarius, Streptococcus thermophilus, and combination thereof.

7. The composition, in accordance with any of clauses 1 to 6, comprising at least one additional component that is selected from the group consisting of Vitamin 1, Vitamin B2, Vitamin B3, Vitamin B6, Vitamin B9, Vitamin B12, Vitamin C, Vitamin E, Zeaxanthin, Hydroxytyrosol, Omega 3 fatty acids (DHA), Glutathione, Copper, Selenium, Zinc, Lutein, and combination thereof.

8. The composition, in accordance with any of clauses 1 to 7, characterized by comprising a relative abundance, as a percentage by weight, comprised of:
4% to 6% of proteins from bacterial lysates of the genus Bacillus;
0.02% to 0.10% of proteins from bacterial lysates of the genus Bifidobacterium;
3% to 8.50% of proteins from bacterial lysates of the genus Lactobacillus;
70% to 99.00% of proteins from bacterial lysates of the genus Saccharomyces;
0.15% to 0.90% of proteins from bacterial lysates of the genus Streptococcus.

9. The use of the composition, in accordance with any of clauses 1 to 8, as a food supplement for the prevention and/or delay of degenerative diseases of the eye.

10. The use of the composition, in accordance with clause 9, where the composition is administered orally in an amount between 100 mg to 400 mg and between 1 to 6 times a day.

11. The use of the composition, in accordance with clauses 9 or 10, where the composition is presented in powder form inside watertight sachets or encapsulated in gelatin capsules.

12. The composition, in accordance with any of clauses 1 to 8, for use as a medicinal product.

13. The composition, in accordance with clause 12, for use in the prevention and treatment of degenerative diseases of the eye.

14. The composition, in accordance with clauses 12 or 13, where the composition is administered orally in an amount between 100 mg to 400 mg of the composition and between 1 to 6 times a day.

15. A pharmaceutical composition comprising an effective pharmaceutical quantity of the composition, in accordance with any of clauses 1 to 8, and a pharmaceutically acceptable excipient.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%”, “80% to 95%” and “90% to 95%”). Certain implementations of the technology are set forth in examples below

### PREFERENTIAL EMBODIMENT OF THE INVENTION

In order to contribute to a better understanding of the invention, and in accordance with a practical realization thereof, we have included as an integral part of this description a preferred embodiment of the present invention.

### Example 1—Preparation of a Lysate Composition of Probiotic Microorganisms

The process consists in obtaining the mixture of dry microbial lysates of different strains of Bacillus sp., Lactobacillus sp., Bifidobacterium sp., Streptococcus sp. and Saccharomyces sp. To achieve this, these are grown by fermentation, concentrated, broken up, and finally the cellular components are dried. The postbiotic formulation will be the mixture of lysates that can be accompanied by food ingredients in a capsule or powder supplement format.

For growth, an inoculum is prepared in a culture medium in a flask of each microorganism, in adequate quantity and volume to be able to proceed to inoculate the fermenter with respective incubated inoculums. To this end, the microorganisms preserved in vials with 20% glycerol at −80° C., are immersed in a water bath at 30° C. to allow a rapid and complete thawing of the respective microorganism.

Once the inocula of the respective microorganisms have been obtained, the fermenters are sown directly separately. The percentage of planting is 3% for Bacillus species and 5% for each species of Bifidobacterium, Lactobacillus, Streptococcus and Saccharomyces.

After sowing the fermenter with the inoculum of the corresponding microorganism, we proceed to the fermentation phase of each of the microorganisms for the generation of microbial biomass, establishing the growth conditions for each species.

For the cultivation of Bacillus, the inoculum is grown in Nutrient Broth (NB) at 30° C. for 15 h at 250 rpm. It is cultured in a fermentation medium composed of Glucose H₂O 25 g/l, Yeast extract 5 g/l, K₂HPO₄ 2.5 g/l, MgSO₄·7H₂O 5 g/l, Tween 80 1 g/l, at 30° C.±1° C. throughout the process and pH 6.8±0.1 by sterile addition of NH₄OH 25% or H₃PO₄ 35% automatically. Dissolved oxygen is maintained ≥40% by agitation (150-250 rpm) and aeration (1-1.5 vvm) with a working pressure of 0.5 atm. After approximately 20-24 h, the base consumption (NH₄OH) is stopped and the temperature is lowered to 4-8° C. to stop cell metabolism and to harvest the broth.

In the case of Streptococcus, the inoculum is grown in Tryptic Soy Broth (TSB) to 37° C. at an agitation of 150 rpm for 24 h. The fermentation medium consists of Glucose 25 g/l, milk powder 8 g/l, Yeast extract 5 g/l, K₂HPO₄ 2 g/l, KH₂PO₄ 2 g/l, MgSO₄·7H₂O 0.5 g/l, (NH₄)SO₄ 0.5 g/l. The growth temperature is 37° C.±1° C. and the pH is maintained at 6.8±0.1 throughout the process by the sterile addition of NH₄OH 25% H₃PO₄ 35% automatically. Agitation is 150 rpm. At the end of the fermentation, after approximately 20-24 h, the temperature is lowered to 4-8° C. to stop the cellular metabolism and to harvest the broth.

The growth of the inoculum of Bifidobacterium and Lactobacillus is made in De Man, Rogosa, Sharpe Broth (MRS Broth) at 37° C. for 24 h. Fermentation is carried out at 37° C.±1° C., Ph 6.2±0.1 and 6.4±0.1, respectively, with the sterile addition of NH₄OH 25% or H₃PO₄ 35% automatically, and 50 rpm (the minimum value that guarantees the correct homogenization of the components of the medium) The culture medium is composed of Glucose H₂O 20 g/l, Yeast extract 5 g/l, K₂HPO₄ 2 g/l, Casein peptone 10 g/l, Milk powder 8 g/l, Sodium acetate 5 g/l, Diammonium citrate 2 g/l, Mn SO₄_{^{−}}H₂O 0.05 g/l, MgSO4 7H₂O 0.2 g/l, Tween 80 1 g/l. Initially nitrogen is bubbled in until the dissolved oxygen concentration is at 0%, repeating the operation whenever necessary to maintain O₂ at this level throughout the process. At the end of the fermentation (20-24 h approx), the temperature is lowered to 4-8° C. to stop the cellular metabolism and to harvest the broth.

The inoculum of S. cerevisiae is grown in YPD for 24 h. In the fermenter the temperature is maintained at 30° C. and Ph 5 at 150 rpm and 1.5 vvm. The fermentation stops in the fermenter itself, lowering the temperature to a value between 4-8° C.

The recovery of the biomass obtained based on the fermentation of each of the microorganisms is carried out by centrifugation or microfiltration, obtaining wet recovered biomasses, concentrating them as many times as necessary to reach between 1.00E+09 to 1.00E+11 cfu/ml in the concentrated biomasses of each microorganism.

Subsequently, the rupture or lysis of the cells of each of the wet recovered biomasses is carried out with heat treatment and sonication, obtaining respective bacterial lysates that are subsequently dried separately in a lyophilizer or atomizer. The dry bacterial lysates are mixed in a mixer, to obtain the modulating composition.

Each batch of viable cell culture undergoes an autoclave sterilization cycle at 121° C. for 20 minutes. Subsequently, they are subjected to sonication for 20 min, at an output power of 500 W with an amplitude of 40% and 10 seconds of pause, obtaining a mass of lysed cells of the probiotic (Qsonica, Q500). The final solution is freeze-dried and ground to obtain a lysate of the powdery probiotic microorganism. The powder is stored in a cool environment away from heat.

For the analysis of the lysates, 0.5 gr of powder stock is taken from each batch and dissolved in 10 ml of distilled water. A dilution 1:8 is prepared from this suspension (100 sample ul and 700 ul water), and centrifuged for 5 minutes at 5000 rpm. The supernatant is collected, and 1.5 ul of sample is placed in a cuvette to measure the absorbance at 260 nm, 230 nm and 280 nm in a Nanodrop Tecan Spark 10M using distilled water as a blank. The measurement is done at least twice, and it is usual to repeat it more than four times.

The peak of maximum absorption for the quantification of nucleic acids, DNA, occurs at the wavelength of 260 nm, therefore, at that wavelength, the absorption will be proportional to the concentration of DNA. (ng/ul DNA: A260 nm×dilution factor×50 (conversion factor)

The amount of DNA present in each of the lysates is as follows:

TABLE 2
DNA concentrations of each of the dry lysates of bacterial strains
that were prepared in accordance with the procedure.

| Probiotic | DNA mg/g | SD |
|---|---|---|
| Lactobacillus casei | 13.68 | 1.89 |
| Lactobacillus acidophilus | 5.44 | 0.85 |
| Lactobacillus fermentum | 3.75 | 1.24 |
| Lactobacillus reuteri | 5.96 | 0.35 |
| Lactobacillus rhamnosus | 14.72 | 1.03 |
| Bifidobacterium. lactis | 3.05 | 0.50 |
| Streptococcus thermophilus | 10.71 | 1.65 |
| Bifidobacterium bifidum | 7.60 | |
| Bacillus subtilis | 6.68 | 0.99 |
| Bacillus licheniformis | 5.75 | 0.87 |
| Saccharomyces cerevisiae | 12.01 | 1.93 |

A sample of the composition object of the invention was prepared, with the following dry bacterial lysates in the following proportions:

TABLE 3
Composition of sample 1 of the composition object
of the invention. The percentage by weight of each
of the genera described in this document is shown.

| | Sample 1 |
|---|---|
| Genera of dry bacterial lysates | (wt %) |
| Genus Bacillus | 16 |
| Genus Lactobacillus | 21 |
| Genus Streptococcus | 3 |
| Genus Saccharomyces | 55 |
| Genus Bifidobacterium | 5 |

In an even more preferred way, and to determine the effectiveness of the composition object of the invention in patients of AMD, a study in patients with this disease has been carried out, in which they were administered a dose of the composition object of the invention of 400 mg/3 times a day.

In particular, they were administered the composition object of the invention comprising dry bacterial lysates of the following species: The quantities are indicated as a percentage by weight:

TABLE 4
Composition of sample 1 of the composition object of the invention.
The percentage by weight of each of the bacterial species comprising
the dry bacterial lysates described in this document is shown.

| | % |
|---|---|
| Species | weight |
| Bacillus licheniformis | 10 |
| Bacillus subtilis | 6 |
| Bifidobacterium bifidum | 1 |
| Bifidobacterium lactis | 4 |
| Lactobacillus acidophilus | 10 |
| Lactobacillus casei | 1 |
| Lactobacillus fermentum | 6 |
| Lactobacillus reuteri | 2 |
| Lactobacillus rhamnosus | 2 |
| Saccharomyces cerevisiae | 55 |
| Streptococcus thermophilus | 3 |

### Capsule 1. Example of Capsules for the Treatment of AMD

Gelatin capsules containing the following components were prepared:

TABLE 5
Example of composition for capsules.

| | | |
|---|---|---|
| Vitamin E | 133.33 | IU |
| Vitamin C | 166.67 | Mg |
| Lutein | 3.33 | Mg |
| Zeaxanthin | 0.67 | Mg |
| Zinc | 8.33 | Mg |
| Copper | 0.67 | Mg |
| Composition of lysates of probiotic microorganisms | 200 | Mg |
| prepared according to example 1 analyzed in example 2 | | |

Patients were given 1 to 3 capsules daily.

### Capsule 2. Example of Capsules for the Treatment of AMD

Gelatin capsules containing the following components were prepared:

TABLE 6
Example of composition for capsules

| | | |
|---|---|---|
| Vitamin E | 133 | IU |
| Vitamin C | 167 | Mg |
| Lutein | 3.3 | Mg |
| Zeaxanthin | 0.67 | Mg |
| Zinc | 8.33 | Mg |
| Copper | 0.67 | Mg |
| Composition of lysates of probiotic microorganisms | 400 | Mg |
| prepared according to example 1 analyzed in example 2 | | |

Patients were given 1 to 3 capsules daily.

### Capsule 3—Example of Capsules for the Treatment of AMD

Gelatin capsules containing the following components were prepared:

TABLE 7
Example of composition for capsules

| | | |
|---|---|---|
| Omega 3 fatty acids (DHA) | 47 | Mg |
| Vitamin B1 | 0.4 | Mg |
| Vitamin B2 | 0.5 | Mg |
| Vitamin B3 | 5 | Mg |
| Vitamin B6 | 0.5 | Mg |
| Vitamin B9 | 67 | μg |
| Vitamin B12 | 0.8 | μg |
| Vitamin C | 27 | Mg |
| Vitamin E | 4 | Mg |
| Hydroxytyrosol | 0.25 | Mg |
| Glutathione | 0.33 | Mg |
| Selenium | 18 | μg |
| Zinc | 2.5 | Mg |
| Copper | 0.3 | Mg |
| Composition of lysates of probiotic microorganisms | 400 | Mg |
| prepared according to example 1 analyzed in example 2 | | |

Patients were given 1 to 3 capsules daily.

### Example 2—Analysis of the Composition Prepared in Example 1

### 2.1. Sample Preparation and Protein Digestion

Dissolve 400 mg of the composition of example 1 (hereinafter, Sample) with a chaotropic buffer containing 8.4 M urea (USB Corporation, Cleveland, OH, USA), 2.4 M thiourea (Sigma Aldrich), 5% CHAPS (Sigma Aldrich), 5 mM TCEP (Sigma Alrich) and a protease inhibitor cocktail (Sigma Aldrich), and incubate on ice for 15 minutes. Homogenization was performed by sonication with ultrasonic application for 5 minutes in Branson 2510 bath (Marshall Scientific, New Hampshire, USA). The homogenate was centrifuged at 20000×g at 4° C. for 10 minutes, and the supernatant containing the solubilized proteins was used for further analysis. Consequently, 40 μg of proteins were precipitated by the methanol/chloroform method and resuspended in 40 μg of a UTT buffer (7M urea, 2M thiourea, 10 mM TEAB (sigma Aldrich) of multichaotropic sample solution.

The resuspended Sample was reduced with 2μ of TCEP 50 mM at 37° C. for 60 minutes, followed by the addition of 1 μL of MMTS reactant (SCIEX, Foster City, CA, USA) cysteine blocker for 10 minutes at room temperature. The Sample was diluted to 140 μl with TEAB mM in order to reduce the urea concentration. Finally, digestion began by adding 2 μg of MS grade Pierce trypsin (Thermo-Fisher Scientific, Inc., Waltham, MA, USA) to each fraction in a ratio of 1:20 (w/w) and incubated at 37° C. overnight in a stirrer. The digested Sample was subjected to evaporation until dryness in a vacuum concentrator.

### 2.2. Liquid Chromatography and Mass Spectrometry Analysis (LC-MS)

The digested Sample was washed/desalted using Stage-Tips with Empore 3M C18 (Sigma Aldrich) discs. A 1 μg aliquot portion of the resulting peptides was subjected to 1D-nano LC ESI-MS/MS (Liquid Chromatography Electrospray Ionization Tandem Mass Spectrometric) analysis using an Eksigent Technologies nanoLC Ultra 1D plus nano-liquid chromatography system (SCIEX, Foster City, CA, USA) coupled to a Triple TOF 5600 (SCIEX) high-speed mass spectrometer with a Nanspray III source. The analysis column used was an Acquity UPLC M-Class Peptide BEH C18 reverse phase silica-based column (Waters Corporation, Milford, MA, USA). The trap column was a C18 Acclaim PepMap™ 100 (Thermo-Fisher Scientific Inc.), 100 μm×2 cm, 5 μm particle diameter, pore size 100 Å, connected in line with the analysis column. The charge pump supplied a 0.1% formic acid solution in water at 2 μl/min. A flow rate of 250 nL was applied by a nano-pump operated under gradient elution conditions. The peptides were separated using a gradient of 250 min with a B phase ranging from 2% to 90% (mobile phase A: 2% acetonitrile (Scharlab, S. L., Spain), 0.1% formic acid (Sigma Aldrich); mobile phase B: 100% acetonitrile, 0.1% formic acid). The injection volume was 5 μl. Data were obtained using an Ionspray floating voltage of 2300 V, curtain gas 35, interface heater temperature 150, source gas 1 25 and degrouper potential 150 V. For IDA (Intelligent Data Analysis) parameters, a 0.25 s MS scan in the mass range of 350 to 1250 Fa was followed by 35 MS/MS scans of 100 ms in the mass range of 100 to 1800. The change criteria were established for ions greater than a mass/charge ratio (m/z) of 350 and less than m/z 1250, with a charge state of 2-5 and an abundance threshold greater than 90 counts/second (cps). The above target ions were excluded for 15 s.

### 2.3. Analysis of Proteomic Data and Search for Sequences

The spectrometric data obtained were processed using the PeakView v2.2 (SCIEX) software and exported to mgf files that were searched using Mascot Server v2.5.1 (Matrix Science, London, United Kingdom) against a protein database containing protein sequences of microorganisms of the genera Bacillus, Lactobacillus, Bifidobacterium, Streptococcus and Saccharomyces from the Uniprot/Swissprot knowledge base (https://www.uniprot.org/statistics/Swiss-Prot—update: 20170412, 2,542,118 protein sequences), along with commonly existing contaminants. Data were obtained from the total number of coincident peptide spectra for a given protein (referred to as Peptide-to-Spectrum-Matching (PSM) and exponentially modified Protein Abundance Index (emPAI)) for each of the proteins. PSM and emPAI, can be used as a relative quantitative result of proteins in a complex mixture based on protein coverage by matching peptides in a database of results. The LC-MS analyses were carried out at the proteomics facilities of the National Center for Biotechnology of the Higher Council for Scientific Research, which are part of ProteoRed.

### 2.4. Protein Abundances and Functions in the Sample

The Sample was thoroughly analyzed in terms of the proteins expressed, and 937 proteins were identified. For the 937 proteins identified, spectral counts were obtained to get an indication of their abundance. Normalized emPAI values (nemPAI %) were obtained based on the emPAI values by dividing each individual value by the sum of all empPAI values and multiplying each resulting value by 100%, to obtain the relative abundance of each protein with respect to the total proteins.

In addition, a functional analysis of the proteins in the Sample was performed by assigning k numbers using the KEGG internal annotation tool for KEGG Orthologies (KO), and searching clusters of orthology groups (COG) to analyze the relative abundance of specific proteins in certain metabolic processes defined in the Kyoto Encyclopedia of Genes and Genomes (KEGG) and COG (http://eggnogdb.embl.de/#/app/home).

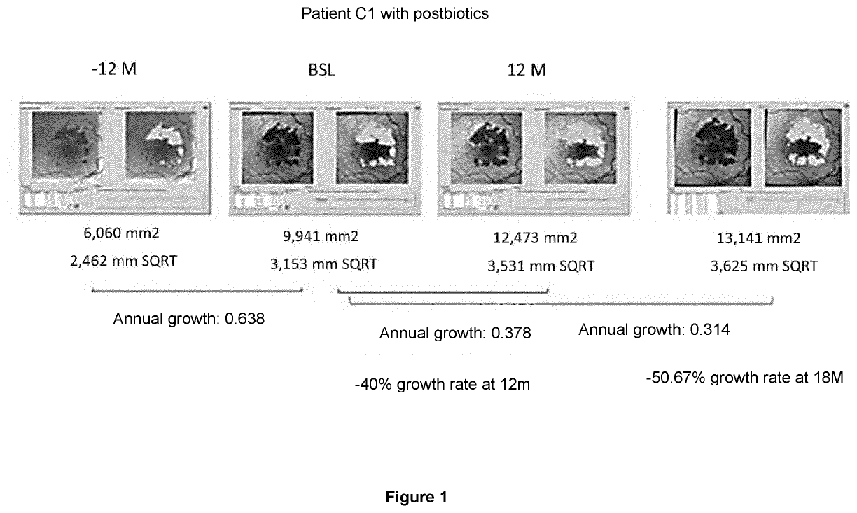

FIG. 1 shows the distribution of nemPAI values. As can be seen, the distribution obtained has an exponential shape which indicates the existence of a great diversity of proteins in the Sample.

The identification of the microbial origin of the Sample was made by a comparative search between the mass spectrometry data of the Sample against the proteins of microorganisms of the genera Bacillus, Lactobacillus, Bifidobacterium, Streptococcus and Saccharomyces of the Uniprot/Swissprot database. The bacterial origins and contribution to the proteome of the Sample are summarized in the following table:

TABLE 8
Mass spectrometry data of the Sample against the proteins of
microorganisms of the genera Bacillus, Lactobacillus, Bifidobacterium,
Streptococcus and Saccharomyces from the Uniprot/Swissprot
database, where the bacterial origins and contribution to the
proteome of the Sample are determined.

| | Number of | Relative number of | Relative protein |
|---|---|---|---|
| Genus | proteins | proteins (%) | abundance (%) |
| Saccharomyces | 727 | 77.59 | 87.63 |
| Lactobacillus | 117 | 112.48 | 4.69 |
| Bacillus | 75 | 8.00 | 4.35 |
| Streptococcus | 10 | 1.07 | 0.33 |
| Bifidobacterium | 3 | 0.32 | 0.06 |
| non-assignable | 5 | 0.53 | 0.06 |
| Total | 937 | 99.98 | 97.13 |

As can be seen, the microorganisms of the genus Saccharomyces are the ones that contribute the most in terms of the total number of proteins and in terms of the level of expression of proteins contained in the Sample. 10 proteins of the 937 identified with relative abundance of 1% or more correspond to this genus. These proteins and their nemPAI values are identified in the table below.

TABLE 9
Ten proteins of the 937 identified in the
Sample with relative abundance of 1%

| AC Protein | nemPAI (%) |
|---|---|
| E7KMZ6 | 4.84 |
| A0A0L8VH73 | 3.37 |
| P0C0X0 | 2.34 |
| A0A0L8VPY0 | 1.76 |
| A0A0L8VLA9 | 1.52 |
| P00359 | 1.26 |
| E7LV64 | 1.16 |
| P22943 | 1.11 |
| P05743 | 1.00 |
| P21306 | 1.00 |

Functional analysis based on KEGG orthology (KO) performed to check the functions of proteins in the different metabolic pathways that are defined by KEGG and COG revealed that 643 of the 937 proteins could be assigned to 352 functional categories. Among the notable functions of the proteins with greater abundance is that represented by the category K01689 (enolase [EC: 4.2.1.11]) to which the protein E7LV64 corresponds. This enolase catalyzes the reversible conversion of 2-phosphoglycerate to phosphoenolpyruvate and is essential to the degradation of carbohydrates by glycolysis. Notable functions in the protein profile include K03530 (histone-like binding protein), K13953 (alcohol dehydrogenase) and K03671 (thioredoxin). All of them have a contribution to total metabolic activity of at least 1%.

### Example 3: Pilot Study to Evaluate the Safety and Efficacy of Oral Postbiotic Therapy in Patients with Secondary Geographic Atrophy in Age-Related Macular Degeneration, Myopia, or Angioid Streaks (Revers)

This example describes a study that meets the following conditions:
Open-label, non-randomized study in patients with macular atrophy
Treatment: postbiotics+antioxidants and vitamins, with the composition of capsule 1

### 3.1 Objective of the Study

To evaluate the growth rate of geographic atrophy according to the transformation of the square root of the area (SQRT) measured in fundus autofluorescence (FAF) with respect to the growth rate of the previous year.
To assess the safety and tolerability of daily dosing of oral postbiotics assessed by incidence and/or clinically significant changes of a combination of ocular and non-ocular adverse events.

### 3.2 Study Design

A retrospective review of data from patients participating in the study was conducted, characterizing the progression of geographic atrophy in patients with age-related macular degeneration and another group of patients with similar lesions who did not receive the treatment. The study followed the principles of the Declaration of Helsinki and was approved by the ethics committee of the Teknon Medical Center. Informed consent was obtained from all participants included in the study after explaining the nature and consequences of the study.
Open-label, non-randomized study in patients with macular atrophy.
Two groups: Ten patients with moderate-rapid growth (>0.2<0.3 SQRTmm/a) with a known rate of progression, obtained with a previous follow-up of 12 months, to subsequently explore whether the rate of progression slows down 12 months after starting treatment (14 eyes, 9 patients, 1 dropout). The same procedure is performed in a group of ten patients with similar lesions, but without any supplementation (13 eyes, 9 patients)

### 3.3 Results

1) The use of oral postbiotics was well tolerated with minimal adverse effects.
2) Progression of atrophy:

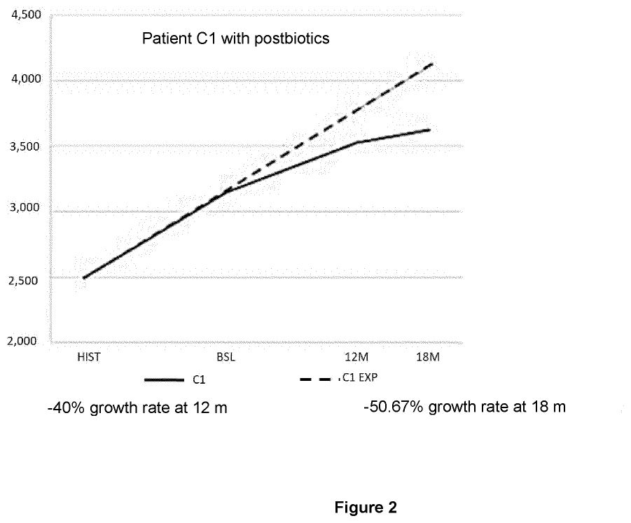

After 12 months of postbiotic treatment with sample 1, on average, the eyes showed a 26.58% reduction in lesion growth measured by SQRT compared to the development of the same lesion during the year prior to the start of treatment. Considering only patients with AMD, 10 eyes of 6 patients 19.87% reduction. (See FIGS. 1 and 2.)

The measurement was performed with two independent gradations using Spectralis Heidelberg's semi-automated region search software.

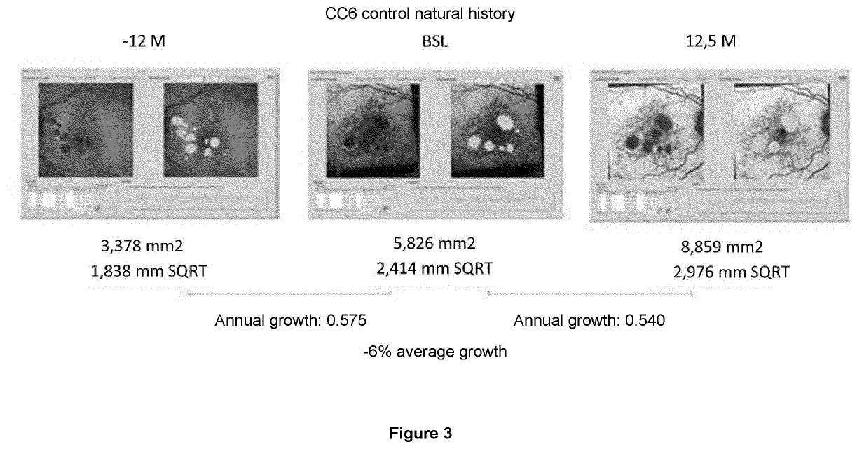

The second group of eyes with similar lesions but without treatment did not show this slowing of progression predicted according to their natural history (See, FIG. 3).

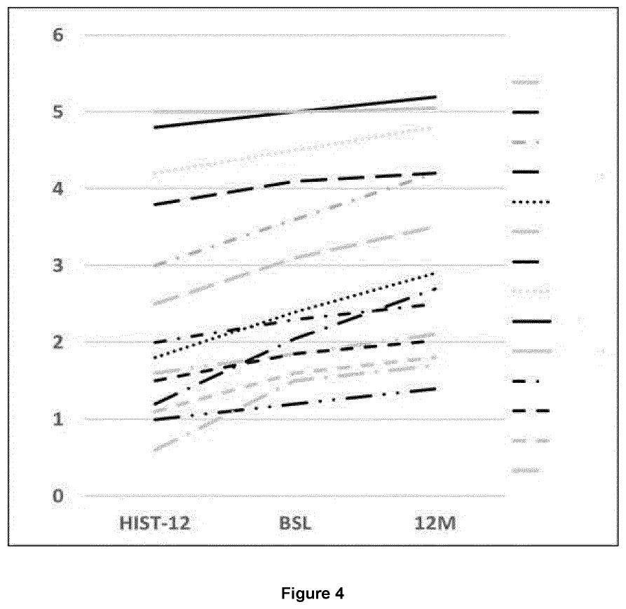

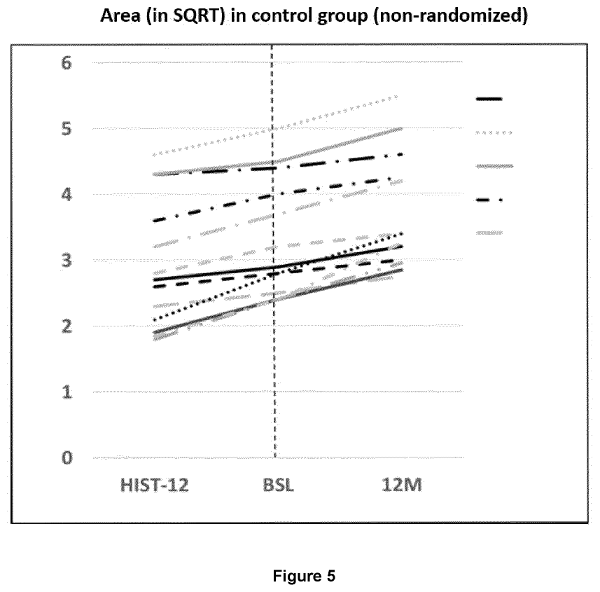

FIGS. 4 and 5 show the areas in SQRT of the groups of patients treated with postbiotics and those not treated, 12 months before and after starting treatment, observing a slowdown of 26.58% and acceleration of 4.5% according to their natural history, respectively.

### 3.4. Conclusions of this Study

In this pilot study, after 12 months of treatment with the postbiotic, the eyes, on average, showed a 26.58% reduction in lesion growth measured by SQRT compared to the growth of the same lesion during the year prior to the start of treatment, while a control group of eyes (non-randomized) showed a 4.5% increase.

### 3.5. Potential Medical and Socioeconomic Impact of the Study

The advanced form of age-related macular degeneration (AMD) is the leading cause of blindness in patients over 50 years of age in developed countries, and the most prevalent atrophic form has no treatment. In atrophic AMD there is the death of retinal tissue affecting the central part of the retina, and this area of destruction grows inexorably, increasing its size progressively and in very few years. Today it is a real epidemic that is increasing exponentially, due to the aging of the population.

Many attempts at clinical trials that have attempted to slow the progression of the disease have failed, due to the very complex multifactorial degenerative character of the disease. Today, a drug based on monthly intravitreal injections is under review by the FDA to assess its approval, with percentages of reduction in the progression of the disease of between 16 and 18% compared to the control group.

The 12-month REVERS pilot study using oral postbiotic therapy in patients with macular atrophy showed an average slowing of progression of 26.58%, and 19.87% in patients with geographically advanced AMD, compared to the progression that the lesion had shown in the previous year. In a group of patients who were not treated, this reduction was not observed, but on average the lesions grew by 4.5%, close to the linear growth that these lesions are known to have. These results would be in the same range of efficacy as the drugs currently under study and in the approval phase, but with an enormous reduction in the medical and socioeconomic impact, due to it being an oral treatment, compared to monthly injections that must be performed for life. It would avoid both the clinical complications associated with invasive, frequent and chronic treatments, as well as the high socioeconomic costs of all kinds, both for the health system and for patients and their families, which entail these expensive, frequent and ongoing therapies for many years in elderly people.

### Example 4: Study of the Immunomodulatory Activity of a Postbiotic Composition of the Invention

During the performance of the study the following reagents and equipment were used:
Fetal bovine serum (FBS). Gibco, 10270-106.
Hank's Salts H-2387.
L-glutamine 200 mM. Sigma, G7513.
Penicilin/streptomicin (P/S). Gibco 15140-122.
Tripsin-EDTA (10×). Sigma T4174.
PBS (10×). ROCHE 11666789001.
MycoAlert® Mycoplasma Detection Kit. Lonza LT07-318.
RPMI-1640. Sigma, R0883.
Human IL-8 ELISA Kit. BD, 555244.
Human TNF alpha ELISA Kit. Invitrogen, EH3TNFA2.
Sodium Dodecyl Sulphate (SDS). CAS No 151-21-3 Sigma, L4509.
E. coli lipopolysaccharide (LPS). Sigma, L3012.
PMA (Phorbol 12-myristate 13-acetate). Sigma, P1785.
β-mercapthoethanol. Sigma; 63689.
Ficoll® Paque Plus Cytiva. Sigma; GE17-1440-02.
Human IL-6 ELISA Kit. Invitrogen; KHC0062.
Automatic pipettes.
Multi-channel pipettes.
Thermostatic bath with stirring capacity.
Freezer −80° C.
Refrigerator.
CO2 incubator.
Laminar flow cabins.
Varioskan Lux micro plate reader (fluorimeter, luminometer and spectrophotometer)
Inverted optical microscope.
Haemocytometer.
Microplate stirrer.
Microcentrifuge.

The postbiotic composition of the invention was freshly prepared on the day of the assay, directly in the culture medium, following the protocol described above. The composition is as follows:

TABLE 10
Composition of sample 2 of the composition object of the invention.
The percentage by weight of each of the bacterial species comprising
the dry bacterial lysates described in this document is shown.

| | % |
|---|---|
| Species | weight |
| Bacillus coagulans | 1 |
| Bacillus mesentericus | 5 |
| Bacillus licheniformis | 5 |
| Bacillus subtilis | 5 |
| Bifidobacterium bifidum | 2.5 |
| Bifidobacterium lactis | 2.5 |
| Lactobacillus acidophilus | 4 |
| Lactobacillus bulgaricus | 4 |
| Lactobacillus casei | 5 |
| Lactobacillus fermentum | 3 |
| Lactobacillus reuteri | 2 |
| Lactobacillus rhamnosus | 3 |
| Saccharomyces cerevisiae | 55 |
| Streptococcus thermophilus | 3 |

THP-1 cells (human monocyte cell line, ATCC® TIB-202) were stored in Gaiker's culture bank and the absence of contaminating mycoplasmas was checked immediately after thawing them. THP-1 cells grow in suspension at 37° C. in a humid atmosphere of 5% CO2. The cells were kept in culture medium (RPMI+10% FBSi+50 μM βmercapthoethanol), for a minimum of two weeks prior to any experiment. Cells were subcultured before reaching 80% confluence (cell/volume concentration).

A cell suspension of 1×10⁵ cells/mL was prepared 24 hours before the performance of the immunomodulatory activity test. 1×10⁴ cells/well were dispensed in 96-well plates in presence of 0.31 μg/mL PMA to differentiate the monocytes to macrophages. Plates were incubated at 37° C., 5% C02 for 24 hours.

For the immunomodulatory assay, PMA-differentiated THP-1 cells were incubated with the postbiotic composition of the invention at 15.63 μg/mL for 24 hours in presence of the inflammatory stimulus, lipopolysaccharide, LPS (20 μg/mL). Non-inflamed cells (cells exposed to culture medium without stimulus) were used as a negative test control. The secretion of IL-6, by cells was measured by ELISA means following manufactures instructions. After that exposure time, cell supernatants were collected and analysed. IL-6 has been reported in the state of art as a key player in AMD progression: Droho, S., Cuda, C. M., Perlman, H. et al. Macrophage-derived interleukin-6 is necessary and sufficient for choroidal angiogenesis. Sci Rep 11, 18084 (2021). https://doi.org/10.1038/s41598-021-97522-x

| IL-6 (pg/mL) | Replicates | | | Average | Desvest |
|---|---|---|---|---|---|
| C− | 0.000 | 0.000 | 0.000 | 0.000 | 0.000 |
| C+ | 990.363 | 1012.901 | 1136.324 | 1046.529 | 78.577 |
| Postbiotic | 940.153 | 821.217 | 870.801 | 877.391 | 59.741 |
| composition of the | | | | | |
| invention (15, 63 | | | | | |
| μg/ml) + LPS | | | | | |

A reduction in the levels of IL-6 is observed compared to LPS alone, this result provides insight about the molecular mechanism underlying the therapeutic effect of the postbiotic composition of the invention in treating age related macular degeneration.

### Example 5: Pilot Study to Evaluate the Safety and Efficacy of Oral Postbiotic Therapy Using a Different Composition

Composition of sample 2 of the composition object of the invention was also tested in patients in an analogous way to Example 3 and the results observed were very similar.

1. A composition for oral administration that comprises lysates of probiotic microorganisms in a form of dry powder that are an amount, in percentage by weight with respect to a total, comprised between:
15% and 20% of bacterial lysates of a genus Bacillus;
15% and 35% of bacterial lysates of a genus Lactobacillus;
1.5% and 8% of bacterial lysates of a genus Streptococcus;
45% and 60% of bacterial lysates of a genus Saccharomyces; and
1.5% and 8% of bacterial lysates of a genus Bifidobacterium,
for use in a prevention or treatment of degenerative diseases of an eye.

2. The composition for use, according to claim 1, wherein the composition comprises lysates of probiotic microorganisms in an amount in percentage by weight on the total lysates of the composition:
approximately 16% of bacterial lysates of the genus Bacillus;
approximately 5% of bacterial lysates of the genus Bifidobacterium;
approximately 21% of bacterial lysates of the genus Lactobacillus;
approximately 55% of yeast lysates of the genus Saccharomyces; and
approximately 3% of bacterial lysates of the genus Streptococcus.

3. The composition for use, according to claim 1, wherein the bacterial lysates of the genus Bacillus are of a species that are selected from the group consisting of Bacillus coagulans, Bacillus lichenformis, Bacillus mesentericus, Bacillus subtilis, Bacillus clausii, Bacillus paralicheniformis, and combinations thereof.

4. The composition for use, according to claim 1, wherein the bacterial lysates of the genus Bifidobacterium are of a species that are selected from the group consisting of Bifidobacterium bifidum, Bifidobacterium lactis, Bifidobacterium breve, Bifidobacterium longum, Bifidobacterium animalis subsp. lactis, Bifidobacterium infantis, Bifidobacterium animalis, and combinations thereof.

5. The composition for use, according to claim 1, wherein the bacterial lysates of the genus Lactobacillus are of a species that are selected from the group consisting of Lactobacillus acidophilus, Lactobacillus bulgaricus, Lactobacillus casei, Lactobacillus fermentum, Lactobacillus reuteri, Lactobacillus rhamnosus, Lactobacillus paracasei, Lactobacillus helveticus, Lactobacillus johnsonii, Lactobacillus plantarum, Lactobacillus salivarius, Lactobacillus gasseri, Lactobacillus kefiri, and combinations thereof.

6. The composition for use, according to claim 1, wherein the bacterial lysates of the genus Saccharomyces are of a species that are selected from the group consisting of Saccharomyces cerevisiae, Saccharomyces boulardii, and combinations thereof.

7. The composition for use, according to claim 1, wherein the bacterial lysates of the genus Streptococcus are of a species that are selected from the group consisting of Streptococcus salivarius, Streptococcus thermophilus, and combinations thereof.

8. The composition for use, according to claim 1, comprising at least one additional component that is selected from the group consisting of Vitamin B1, Vitamin B2, Vitamin B3, Vitamin B6, Vitamin B9, Vitamin B12, Vitamin C, Vitamin E, Zeaxanthin, Hydroxytyrosol, Omega 3 fatty acids (DHA), Glutathione, Copper, Selenium, Zinc, Lutein, and combinations thereof.

9. The composition for use, according to claim 2, comprising a relative abundance, as a percentage by weight, comprised between:
4% to 6% of proteins from bacterial lysates of the genus Bacillus;
0.02% to 0.10% of proteins from bacterial lysates of the genus Bifidobacterium;
3% to 8.50% of proteins from bacterial lysates of the genus Lactobacillus;
70% to 99.00% of proteins from bacterial lysates of the genus Saccharomyces; and
0.15% to 0.90% of proteins from bacterial lysates of the genus Streptococcus.

10. The composition for use, according to claim 1, whereby the composition is administered orally in an amount between 100 mg to 400 mg of the composition and between 1 to 6 times a day.

11. A pharmaceutical composition comprising an effective pharmaceutical amount of the composition, according to claim 1, and a pharmaceutically acceptable excipient for use in the prevention and treatment of degenerative diseases of the eye.

12. The composition for use, according to claim 1, whereby the composition is administered orally in an amount between 100 mg to 400 mg of the composition and between 1 to 6 times a day, wherein the degenerative disease of the eye is age-related macular degeneration.
